SEQUENCE LISTING (7 sequences)

SEQ ID NO: 1 - protein, 372 aa, Rhodotorula taiwanensis
MAPSKRVVVLGSGVVGLSSALTLAQKGYSVHVVARDLPEDTVAQTFASPWAGANWTPFMSKEDGPRQAKWETATFNQWVDLVPQGLAMWLKGTRRYAQDEAGLLGHWYQHITPNYRKLESSECPPGAIGVTYDTLSVNAPKFCQYLQREAQKLGVTFERRLVTSLEQIEGGFDLIVNATGLGAKSIAGVEDQEVEPIRGQTVLVKSDCKRCTMDSSDPNSPAYIIPRPGGEVICGGTYLVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 2 - protein, 372 aa, unidentified
MAPSKRVVVLGSGVVGLSSALTLAQKGYSVHVVARDLPEDTVAQTFASPWAGAVWTPHMSKEDGPRQAKWETATFNQWVDLVPQGLAMWLKGTRRYAQDEAGLLGHWYQHITPNYRKLESSECPPGAIGVTYDTLSVNAPKFCQYLQREAQKLGVTFERRLVTSLEQIEGGFDLIVNATGLGAKSIAGVEDQEVEPIRGQTVLVKSDCKRCTSDSSDPNSPAYIIPRPGGEVICGGTYLVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 3 - protein, 372 aa, unidentified
MAPSKRVVVLGSGVVGLSSALTLAQKGYSVHVVARDLPEDTVAQTFASPWAGAVWTPHMSKEDGPRQAKWETATFNQWVDLVPQGLAMWLKGTRRYAQDEAGLLGHWYQHITPNYRKLESSECPPGAIGVTYDTLSVNAPKFCQYLQREAQKLGVTFERRLVTSLEQIEGGFDLIVNATGLGAKSIAGVEDQEVEPIRGQTVLVKSDCKRCTSDSSDPNSPAYIIPRPGGEVICGGTYGVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 4 - protein, 372 aa, unidentified
MAPSKRVVVLGSGVVGLSSALTLAQKGYSVHVVARDLPEDTVAQTFASPWAGAVWTPHMSKEDGPRQAKWETLTFNWWMDLVPQGLAMWLKGTRRYAQDEAGLLGHWYQHITPNYRKLESSECPPGAIGVTYDTLSVNAPKFCQYLMREAQKLGVTFERRLVTSLEQIEGGFDLIVNATGLGAKMIAGVEDQEVEPIRGQTVLVKSDCKRCTSDSSDPNSPAYIIPRPGGEVICGGTYGVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 5 - protein, 372 aa, unidentified
MAPSKRVVVLGSGVVGLSSALTLAQKGYSVHVVARDLPEDTVSQMFASPWAGAVWTPHMSKEDGPRQAKWETATFNQWVDLVPQGLAMWLKGTRRYAQDEAGLLGHWYQHITPNYRKLESSECPPGAIGVTYDTLSVNAPKFCQYLQREAQKLGVTFERRLVTSLEQIEGGFDLIVNATGLGAKSIAGVEDQEVEPIRGQTVLVKAPCKRCTSDFSDPNSPAYIIPRPGGEVICGGTYGVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 6 - protein, 372 aa, unidentified
MAPSKRVVVLGSGVVGLSSALTLAQKGYSVHVVARDLPEDTVAQTFASPWAGAVWTPHMSKEDGPRQAKWETATFNQWVDLVPQGLAMWLKGTRRYAQDEAGLLGHWYQHITPNYRKLESSPCPPGAIGVTFDTLSVNAPKFCQYLQREAQKLGVTFERRLVTSLEQIEGGFDLIVNATGLGAKSIAGVEDQEVYPIRGQTVLVKSDCKRCTSDSSDPNSPAYIIPRPGGEVILGGTYGVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

SEQ ID NO: 7 - protein, 372 aa, unidentified
MAPSKRVVVLGSGVVGLSSALTLAQKGYSVHVVARDLPEDTVSQMFASPWAGAVWTPHMSKEDGPRQAKWETLTFNWWMDLVPQGLAMWLKGTRRYAQDEAGLLGHWYQHITPNYRKLESSPCPPGAIGVTFDTLSVNAPKFCQYLMREAQKLGVTFERRLVTSLEQIEGGFDLIVNATGLGAKMIAGVEDQEVYPIRGQTVLVKAPCKRCTSDFSDPNSPAYIIPRPGGEVILGGTYGVGNYDLSVDPPTINRILQHCLRLDPSISTDGTLEGIEIVRHNVGLRPARRGGPRVEVERVAFPLERGKSKLSLGTARADSSKPRREVPVVHAYGFSSAGYQQGWGAALEVAELVEGAIGAAPARSSHRWLSKL

(19) United States
(12) Patent Application Publication
(10) Pub. No.: US 2025/0163390 A1
(43) Pub. Date: May 22, 2025

(54) D-AMINO ACID OXIDASE MUTANT AND APPLICATION THEREOF IN PREPARATION OF L-PHOSPHINOTHRICIN

(71) Applicant: ZHEJIANG UNIVERSITY OF TECHNOLOGY, Hangzhou (CN)

(72) Inventors: Yaping Xue, Hangzhou (CN); Liuyu Wang, Hangzhou (CN); Heng Tang, Hangzhou (CN); Yuguo Zheng, Hangzhou (CN)

(21) Appl. No.: 18/840,224

(22) PCT Filed: Sep. 12, 2023
(86) PCT No.: PCT/CN2023/118314

§ 371 Date: Aug. 21, 2024

(30) Foreign Application Priority Data
Dec. 5, 2022 (CN) 202211546961.3

(51) Int. Cl. C12N 9/06; C12P 13/04

(52) U.S. Cl. CPC C12N 9/0024; C12P 13/04; C12Y 104/03003

(57) ABSTRACT

A D-amino acid oxidase mutant with significantly improved catalytic performance, a gene encoding the gene, a vector containing the gene, a genetically engineered bacterium, and the application of the said mutant in the microbial catalytic preparation of L-ammonium glufosinate. The D-amino acid oxidase mutant was obtained from the amino acid shown in SEQ ID NO.1 by single-point mutation or multi-point combined mutation. The beneficial effects are mainly reflected in the following: the D-amino acid oxidase mutant with improved enzyme activity and thermal stability can be used in the microbial catalysed preparation of L-ammonium glufosinate, which is conducive to industrial production and has a better application prospect.

### CROSS REFERENCE TO RELATED APPLICATIONS

This is a 371 application of PCT International Application No. PCT/CN2023/118314, filed on Sep. 12, 2023, which claims priority to Chinese Patent Application No. 2022115469613, filed on Dec. 5, 2022; the disclosures of which are incorporated herein by reference in their entireties.

### TECHNICAL FIELD

The present disclosure relates to a D-amino acid oxidase mutant (DAAO), an encoding gene, a vector and genetically engineered bacteria containing the encoding gene, and an application of the D-amino acid oxidase mutant in preparation of L-phosphinothricin through microbial catalysis.

### BACKGROUND

Phosphinothricin (PPT), with a chemical name of 2-amino-4-[hydroxy(methyl) phosphonyl]butyric acid, is the world's second largest herbicide to which transgenic crops are tolerant. It exhibits excellent herbicidal properties and minimal phytotoxic side effects, possessing significant market potential in the coming years.

There are two optical isomers of PPT, namely L-PPT and D-PPT, but only the L-PPT has herbicidal activity. Generally, the PPT sold in the market is a racemic mixture. If PPT products are used in the form of a pure optical isomer of the L-PPT, a usage amount of the PPT may be significantly reduced, which is of great significance in improving atomic economy, reducing use cost and alleviating pressure on environment.

At present, the most widely used method for the removal of the D-PPT is to use the D, L-PPT as raw materials; the D-PPT is catalyzed by DAAO to obtain an L-PPT precursor 2-carbonyl-4-[hydroxy(methyl)phosphonyl]butyric acid (PPO), which is then catalyzed by amino acid dehydrogenase or transaminase to obtain the L-PPT. Due to the high selectivity and specificity of the DAAO, such method can effectively remove the D-PPT, a key intermediate PPO of the DAAO may also be further converted into the L-PPT, thus effectively improving atomic utilization. Therefore, it is particularly important to screen high-yield PPO mutants.

### SUMMARY

The objective of the present disclosure is to provide a D-AAO mutant with obviously improved catalytic performance, an encoding gene, a vector and genetically engineered bacteria containing the encoding gene, and an application of the mutant in preparation of L-PPT through microbial catalysis to overcome the defects of DAAO, such as low enzyme activity and poor enzyme thermal stability in the prior art.

The technical solutions of the present disclosure are as follows:

A DAAO mutant is obtained by carrying out single-point mutation or multi-point combined mutation at positions 54, 58, 213, 239, 73, 77, 79, 147, 185, 43, 45, 206, 207, 215, 122, 132, 195, and 234 of an amino acid sequence as shown in SEQ ID NO. 1.

The amino acid sequence of the D-AAO as shown in SEQ ID NO. 1 may be referred to as a wild-type enzyme of DAAO in the present application. The wild-type enzyme may have a nucleotide sequence as shown in SEQ ID NO. 8. SEQ ID NO. 1 is an amino acid sequence annotated as the DAAO derived from Rhodotorula taiwanensis. SEQ ID NO. 8 is a nucleotide sequence annotated as the DAAO derived from Rhodotorula taiwanensis.

Preferably, the DAAO mutant includes one of the following mutations: (1) mutation of an amino acid residue N at the position 54 to V, D or T, mutation of an amino acid residue F at the position 58 to H, R, K or Q, and mutation of an amino acid residue M at the position 213 to S, N or R; (2) mutation of the amino acid residue N at the position 54 to V, D or T, mutation of the amino acid residue F at the position 58 to H, R, K or Q, mutation of the amino acid residue M at the position 213 to S, N or R, and mutation of an amino acid residue L at the position 239 to E, D, G or Q; (3) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at the position 239 to E, D, G or Q, mutation of an amino acid residue A at the position 73 to L, mutation of an amino acid residue Q at the position 77 to W, mutation of an amino acid residue V at the position 79 to M, mutation of an amino acid residue Q at the position 147 to M, and mutation of an amino acid residue S at the position 185 to M; (4) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at the position 239 to E, D, G or Q, mutation of an amino acid residue A at the position 43 to S, mutation of an amino acid residue T at the position 45 to M, mutation of an amino acid residue S at the position 206 to A, mutation of an amino acid residue D at the position 207 to P, and mutation of an amino acid residue S at the position 215 to F; and (5) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at the position 239 to E, D, G or Q, mutation of an amino acid residue E at the position 122 to P, mutation of an amino acid residue Y at the position 132 to F, mutation of an amino acid residue E at the position 195 to Y, and mutation of an amino acid residue C at the position 234 to L.

More preferably, an amino acid sequence of the mutant is shown in one of SEQ ID NO. 2 to SEQ ID NO. 7.

SEQ ID NO. 2 is an amino acid sequence of a DAAO mutant I (M213S/F58H/N54V).

SEQ ID NO. 3 is an amino acid sequence of a DAAO mutant II (M213S/F58H/N54V/L239G).

SEQ ID NO. 4 is an amino acid sequence of a DAAO mutant III (M213S/F58H/N54V/L239G/A73L/Q77W/V79M/Q147M/S185M).

SEQ ID NO. 5 is an amino acid sequence of a DAAO mutant IV (M213S/F58H/N54V/L239G/A43S/T45M/S206A/D207P/S215F).

SEQ ID NO. 6 is an amino acid sequence of a DAAO mutant V (M213S/F58H/N54V/L239G/E122P/Y132F/E195Y/C234L).

SEQ ID NO. 7 is an amino acid sequence of a DAAO mutant VI (M213 S/F58H/N54V/L239G/A73L/Q77W/V79M/Q147M/S185M/A43S/T45M/S206A/D207P/S215F/E122P/Y132F/E195 Y/C234L).

The present disclosure further relates to a gene encoding the DAAO mutant and a recombinant vector.

The recombinant vector may be constructed by ligating a nucleic acid of the gene encoding the DAAO mutant to various appropriate vectors through a conventional method in the art. The vector may be selected from various conventional vectors in the art, such as commercially available plasmids, cosmids, phages, or viral vectors, provided that the recombinant expression vector may normally replicate in a corresponding expression host and express the DAAO. Preferably, the vector is the plasmid, and more preferably, a plasmid pET28a.

The present disclosure further relates to genetically engineered bacteria containing the gene encoding the DAAO mutant.

A recombinant expression transformant may be prepared by transforming the constructed recombinant expression vector into a host cell. The host cell is selected from various conventional host cells in the art, provided that the recombinant expression vector may stably self-replicate and effectively express a target protein through an induction of an inducer. According to the present disclosure, preferably, Escherichia coli is selected as a host cell, and more preferably, E. coli BL21 (DE3) is used for the high-efficiency expression of the DAAO mutant of the present disclosure.

The present disclosure further relates to an application of the DAAO mutant in preparation of L-PPT through microbial catalysis.

A reaction formula involved is as follows:

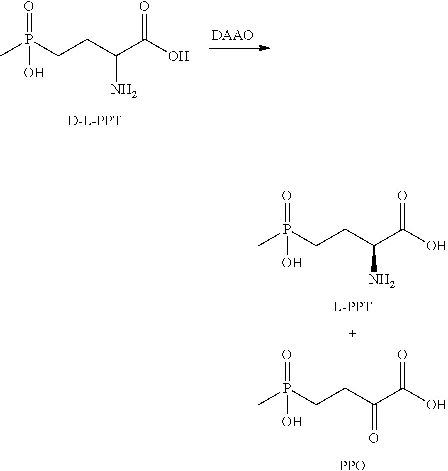

The present disclosure has the beneficial effects that the enzyme activity and the enzyme thermal stability are both improved, and the DAAO mutant can be used for preparing the L-PPT through microbial catalysis, which facilitates industrial production. The DAAO mutant has good application prospects.

### DETAILED DESCRIPTION OF THE EMBODIMENTS

The present disclosure will be further described below with reference to specific examples, but the present disclosure is not limited to the following examples:

A plasmid extraction kit and a DNA purification and recovery kit used in examples are purchased from Hangzhou Qingke Zixi Biotechnology Co., Ltd.; a one-step cloning kit is purchased from Vazyme Co. Ltd.; E. coli BL21 (DE3), plasmid pET-24a (+), etc. are purchased from Sangon Biotech (Shanghai) Co., Ltd., and total gene synthesis is performed by Sangon Biote (Shanghai) Co., Ltd.; DNA labeling, low molecular weight standard protein, and protein precast gel are purchased from Beijing GenStar Co., Ltd.; ClonExpress II One Step Cloning Kit is purchased from Nanjing Vazyme Biotech Co., Ltd; pfu DNA polymerase and Dpn I endonuclease are purchased from Thermo Fisher Scientific (China) Co., Ltd.; and primer synthesis and sequencing are performed by Hangzhou Qingke Zixi Biotechnology Co., Ltd. The use of the above reagents refers to the product manuals.

A reagent in a downstream catalytic process includes D, L-PPT purchased from Sigma-Aldrich Co., Ltd.; 2,4-dinitrophenylhydrazine (DNPH) is purchased from Aladdin Reagent (Shanghai, China), and commoditized micrococcal catalase is purchased from Sigma Aldrich (Shanghai, China). Other commonly used reagents are purchased from Sinopharm Chemical Reagent Co., Ltd.

Other experiment methods without indicating the specific conditions in the following examples are selected based on the conventional methods and conditions or the product manuals.

Products are detected and analyzed through high-performance liquid chromatography (HPLC) in the following examples.

An HPLC analysis method is as follows: a chromatographic column is Welchrom®C18, a column temperature is 30° C., a flow rate is 1 mL/min, a detection wavelength is 232 nm, and a mobile phase is 50 mM (NH₂)HPO₄. 1% of 10% aqueous solution of tetrabutylammonium bromide is added, pH is adjusted to 3.8 with phosphoric acid, and 12% acetonitrile is added.

The content of two conformations of PPT is detected by chiral HPLC analysis. Specifically, in the chiral HPLC analysis, a chromatographic column is Pntulips QS-C18, a mobile phase is 50 mM ammonium acetate solution:methanol=9:1, a detection wavelength is 338 nm, a flow rate is 1 mL/min, and a column temperature is 30° C.

Derivatization reagent: 0.1 g of o-phthalaldehyde and 0.12 g of N-acetyl-L-cysteine are weighed, 10 ml of ethanol is added therein for dissolution promotion, and then, 40 ml of 0.1 M boric acid buffer (pH 9.8) is added. The mixture is fully dissolved by shaking and stored in a refrigerator at a temperature of 4° C. for later use (within 3 days). Derivatization reaction and determination: To 200 μL of sample, 400 μL of derivatization reagent is added, a mixture is mixed well and maintained at 30° C. for 5 minutes, 400 μL of ultrapure water is added to be mixed, and analysis is performed by injecting 10 μL of mixture.

### Example 1: Preparation of Genetically Engineered Bacteria

After total gene synthesis of a gene sequence of wild-type DAAO (wtDAAO) (GenBank number: POY70719.1, with an amino acid sequence as shown in SEQ ID NO. 1 and a nucleotide sequence as shown in SEQ ID NO. 8) derived from Rhodotorula taiwanensis, pET-24a (+)-DAAO was obtained by inserting an expression plasmid pET-24a (+). After sequencing verification, the pET-24a-DAAO was transfected into E. coli BL21 (DE3) of an expression host for the subsequent expression of a recombinant enzyme.

Composition of an LB liquid medium: 10 g/L peptone, 5 g/L yeast powder, and 10 g/L NaCl were dissolved with water and diluted to a final volume, and sterilized at 121° C. for 20 minutes for later use.

After resuscitating the engineered bacteria verified by sequencing, as described above, by streaking on a plate, a single colony was picked and inoculated into 10 mL of LB liquid medium containing 50 g/mL kanamycin and incubated at 37° C. for 10-12 hours with shaking. Then the inoculation broth was transferred to 100 mL of fresh LB liquid medium containing 50 g/mL kanamycin at an inoculum amount of 2%, and was incubated at 37° C. with shaking until OD₆₀₀ reached about 0.8, followed by cooling to 30° C. IPTG was added to the LB liquid medium at a final concentration of 0.5 mM, and inducing incubation continued for 16 hours. At the end of incubation, the incubation broth was centrifuged at 8,000 rpm for 10 minutes, a supernatant was discarded, and cells were harvested and stored in a refrigerator at −20° C. for later use. The cells harvested at the end of incubation were washed twice with 50 mM phosphoric acid buffer (pH 8.0) and then resuspended in 50 mL of phosphoric acid buffer (pH 8.0), followed by lysing homogeneously the cells. A lysis solution was centrifuged to remove cell debris, and a crude enzyme solution containing a recombinant wtDAAO enzyme was obtained.

### Example 2: Construction of DAAO Mutant I (at Positions 54, 58 and 213)

Mutations at positions 213, 58 and 54 were found on the basis of the wild-type DAAO sequence as described in Example 1. Primer sequences for PCR designed for the mutant with mutations at positions 213, 58 and 54 of a mutated DAAO sequence according to the mutation order of mutation sites were shown in Tables 1, 2, and 3.

TABLE 1

| Serial | Primer | |
|---|---|---|
| Number | Name | Primer Sequence |
| 1 | 213A F | TGCAAACGTTGCACCGCTGACAGCAGCGAT |
| 2 | 213R F | TGCAAACGTTGCACCCGTGACAGCAGCGAT |
| 3 | 213N F | TGCAAACGTTGCACCAACGACAGCAGCGAT |
| 4 | 213D F | TGCAAACGTTGCACCGATGACAGCAGCGAT |
| 5 | 213C F | TGCAAACGTTGCACCTGTGACAGCAGCGAT |
| 6 | 213Q F | TGCAAACGTTGCACCCAAGACAGCAGCGAT |
| 7 | 213E F | TGCAAACGTTGCACCGAAGACAGCAGCGAT |
| 8 | 213G F | TGCAAACGTTGCACCGGTGACAGCAGCGAT |
| 9 | 213H F | TGCAAACGTTGCACCCATGACAGCAGCGAT |
| 10 | 213I F | TGCAAACGTTGCACCATCGACAGCAGCGAT |
| 11 | 213L F | TGCAAACGTTGCACCTTGGACAGCAGCGAT |
| 12 | 213K F | TGCAAACGTTGCACCAAGGACAGCAGCGAT |
| 13 | 213F F | TGCAAACGTTGCACCTTCGACAGCAGCGAT |
| 14 | 213P F | TGCAAACGTTGCACCCCTGACAGCAGCGAT |
| 15 | 213S F | TGCAAACGTTGCACCTCTGACAGCAGCGAT |
| 16 | 213T F | TGCAAACGTTGCACCACTGACAGCAGCGAT |
| 17 | 213W F | TGCAAACGTTGCACCTGGGACAGCAGCGAT |
| 18 | 213Y F | TGCAAACGTTGCACCTACGACAGCAGCGAT |
| 19 | 213V F | TGCAAACGTTGCACCGTTGACAGCAGCGAT |
| 20 | 213 R | GGTGCAACGTTTGCAATCGCTCTTCACCAG |

TABLE 2

| Serial | Primer | |
|---|---|---|
| Number | Name | Primer Sequence |
| 1 | 58A F | GGTGCGAATTGGACCCCGGCTATGAGCAAGGAA |
| 2 | 58R F | GGTGCGAATTGGACCCCGCGTATGAGCAAGGAA |
| 3 | 58N F | GGTGCGAATTGGACCCCGAATATGAGCAAGGAA |
| 4 | 58D F | GGTGCGAATTGGACCCCGGATATGAGCAAGGAA |
| 5 | 58C F | GGTGCGAATTGGACCCCGTGTATGAGCAAGGAA |
| 6 | 58Q F | GGTGCGAATTGGACCCCGCAAATGAGCAAGGAA |
| 7 | 58E F | GGTGCGAATTGGACCCCGGAAATGAGCAAGGAA |
| 8 | 58G F | GGTGCGAATTGGACCCCGGGTATGAGCAAGGAA |
| 9 | 58H F | GGTGCGAATTGGACCCCGCATATGAGCAAGGAA |
| 10 | 58I F | GGTGCGAATTGGACCCCGATTATGAGCAAGGAA |
| 11 | 58L F | GGTGCGAATTGGACCCCGTTAATGAGCAAGGAA |
| 12 | 58K F | GGTGCGAATTGGACCCCGAAAATGAGCAAGGAA |
| 13 | 58M F | GGTGCGAATTGGACCCCGATGATGAGCAAGGAA |
| 14 | 58P F | GGTGCGAATTGGACCCCGCCTATGAGCAAGGAA |
| 15 | 58S F | GGTGCGAATTGGACCCCGTCTATGAGCAAGGAA |
| 16 | 58T F | GGTGCGAATTGGACCCCGACTATGAGCAAGGAA |
| 17 | 58W F | GGTGCGAATTGGACCCCGTGGATGAGCAAGGAA |
| 18 | 58Y F | GGTGCGAATTGGACCCCGTATATGAGCAAGGAA |
| 19 | 58V F | GGTGCGAATTGGACCCCGGTTATGAGCAAGGAA |
| 20 | 58 R | GGGTCCAATTCGCACCCGCCCA |

TABLE 3

| Serial | Primer | |
|---|---|---|
| Number | Name | Primer Sequence |
| 1 | 54A F | TGGGCGGGTGCGGCTTGGACCCCGCAAAT |
| 2 | 54R F | TGGGCGGGTGCGCGTTGGACCCCGCAAAT |
| 3 | 54D F | TGGGCGGGTGCGGATTGGACCCCGCAAAT |
| 4 | 54C F | TGGGCGGGTGCGTGTTGGACCCCGCAAAT |
| 5 | 54Q F | TGGGCGGGTGCGCAATGGACCCCGCAAAT |
| 6 | 54E F | TGGGCGGGTGCGGAATGGACCCCGCAAAT |
| 7 | 54G F | TGGGCGGGTGCGGGTTGGACCCCGCAAAT |
| 8 | 54H F | TGGGCGGGTGCGCATTGGACCCCGCAAAT |
| 9 | 54I F | TGGGCGGGTGCGATTTGGACCCCGCAAAT |
| 10 | 54L F | TGGGCGGGTGCGTTATGGACCCCGCAAAT |
| 11 | 54K F | TGGGCGGGTGCGAAATGGACCCCGCAAAT |
| 12 | 54M F | TGGGCGGGTGCGATGTGGACCCCGCAAAT |
| 13 | 54F F | TGGGCGGGTGCGTTTTGGACCCCGCAAAT |
| 14 | 54P F | TGGGCGGGTGCGCCTTGGACCCCGCAAAT |
| 15 | 54S F | TGGGCGGGTGCGTCTTGGACCCCGCAAAT |
| 16 | 54T F | TGGGCGGGTGCGACTTGGACCCCGCAAAT |
| 17 | 54W F | TGGGCGGGTGCGTGGTGGACCCCGCAAAT |
| 18 | 54Y F | TGGGCGGGTGCGTATTGGACCCCGCAAAT |
| 19 | 54V F | TGGGGGGTGCGGTTTGGACCCCGCAAAT |
| 20 | 54 R | CGCACCCGCCCACGGGCTCGCAAAGGTTT |

A PCR (25 L) amplification system was as follows:

12.5 μL of 2×PCR buffer, 0.5 μL of forward primer, 0.5 μL of reverse primer, 0.5 μL of template plasmid, 0.5 μL of dNTP, 0.5 μL of high-fidelity enzyme, and ddH₂O was added to make up to 25 μL.

A PCR amplification procedure was as follows:

(1) pre-denaturation at 95° C. for 5 minutes, (2) denaturation at 95° C. for 30 seconds, (3) annealing at 60° C. for 30 seconds, (4) extension at 72° C. for 5 minutes for 30 cycles, (5) extension at 72° C. for 10 minutes, and (6) storage at 4° C.

At the end of PCR, 5 μL of amplification product was taken for nucleic acid gel electrophoresis analysis, and the PCR product with a clear target band was obtained. 0.5 μL of Dpn I endonuclease was added to the PCR product, and a template was digested at a temperature of 37° C. for 1 hour. After the reaction, the digested product was transformed into BL21 competent cells, and the transformed cells were spread on an LB medium containing 50 g/mL kanamycin and incubated overnight at a temperature of 37° C. Cells were harvested, and transformants containing the mutant were obtained. The cells were obtained as described in Example 1.

### Example 3: High-Throughput Screening of Mutant Library

Screening was performed according to the following experimental steps:

The transformants obtained in Example 2 were inoculated in a 96-well plate, and the plate was then incubated in a constant temperature shaker at a temperature of 37° C. for 12-16 hours, with a revolving speed of the shaker set at 200 rpm. A seed incubation broth of the 96-well plate was transferred to a 96-well plate fermentation medium. In the case of OD₆₀₀=0.4-0.7, an IPTG inducer was added to the 96-well plate fermentation medium, and the incubation broth was incubated in the constant temperature shaker at a temperature of 28° C. for 12-16 hours, with a revolving speed of the shaker set at 200 rpm. The incubated 96-well fermentation broth was centrifuged at 4000 rpm for 10 minutes, a supernatant was discarded, and cells were harvested. The harvested cells were allowed to react with D, L-PPT with a certain concentration in the 96-well plate for 1 hour, and then centrifuged at 4,000 rpm for 10 minutes.

Color development reaction: A multichannel pipette was used to pipette a supernatant of the centrifuged reaction mixture into a 96-well clear plate. A 2 mM 2,4-dinitrophenylhydrazine reagent was added, blown and mixed well with the multichannel pipette, and incubated in a microplate reader thermostatic bath at a temperature of 37° C. for 20 minutes. At the end of the reaction, 100 μL of 1 M NaOH was added, and mixed well for 30 seconds to generate a reddish-brown compound, and an absorbance was measured at 380 mm with a microplate reader. With wtDAAO as a control, positive clones (M213N, M213R, and M213S), (M213S/F58H, M213S/F58R, M213S/F58K, and M213S/F58Q), and (M213S/F58H/N54V, M213S/F58H/N54D, and M213S/F58H/N54T) were obtained through screening.

### Example 4: Comparison of Enzyme Activity of DAAO Mutant I

The positive clones (M213N, M213R, and M213S), (M213S/F58H, M213S/F58R, M213S/F58K, and M213S/F58Q), and (M213S/F58H/N54V, M213S/F58H/N54D, and M213S/F58H/N54T) screened in Example 3 were re-screened, and a re-screening reaction was performed by detecting the catalytic efficiency of the mutant. Specifically, the catalytic efficiency of DAAO was compared with the catalytic efficiency of the DAAO mutant by determining a production of PPO through the HPLC method. A reaction system (1 ml) included: 50 mM racemic PPT ammonium salt, 50 mM phosphate buffer (pH 8.0), 8000 U/L catalase, and 50 g/L DAAO or lyophilized cells of the DAAO mutant. After the reaction was performed for 2 hours, a reaction solution sample was taken and processed, a concentration of PPO was determined and a conversion rate of PPO was calculated (the concentration of PPO product/concentration of initial substrate D, L-PPT×100%), as shown in Table 4.

TABLE 4

| Enzyme | | Product | Conversion |
|---|---|---|---|
| No. | Wild-type/Mutant | PPO/mM | Rate (%) |
| 1 | wtDAAO | 0 | 0.12 |
| 2 | M213S | 0.287 ± 0.05 | 1.15 |
| 3 | M213R | 0.126 ± 0.02 | 0.5 |
| 4 | M213N | 0.189 ± 0.03 | 0.75 |
| 5 | M213S/F58H | 3.58 ± 0.05 | 14.38 |
| 6 | M213S/F58R | 2.74 ± 0.03 | 10.96 |
| 7 | M213S/F58K | 2.69 ± 0.02 | 10.76 |
| 8 | M213S/F58Q | 1.62 ± 0.03 | 6.32 |
| 9 | M213S/F58H/N54V | 8.35 ± 0.04 | 33.4 |
| 10 | M213S/F58H/N54D | 7.68 ± 0.05 | 30.72 |
| 11 | M213S/F58H/N54T | 8.13 ± 0.02 | 32.52 |

### Example 5: Construction of DAAO Mutant (at Position 239)

Mutation of error-prone PCR (epPCR) was performed on the basis of the mutated sequence (N54V/F58Q/M213S) as described in Example 4. Primer sequences were designed for the error-prone PCR, as shown in Table 5:

TABLE 5

| Serial | Primer | |
|---|---|---|
| Number | Name | Primer Sequence |
| 1 | epPCR-F | ATGGCGCCGAGCAAGCGTGTGGTTGTG |
| 2 | epPCR-N | TCAGTGGTGGTGGTGGTGGTGCTCGAGT |

An amplification system of epPCR (30 L) was as follows: Using an instant error-prone PCR kit, amplification was performed in a reaction mixture containing 10 mM Tris-HCl (pH 8.3), 50 mM KCl, 2 mM MgCl₂, and 0.25 mM MnCl₂, with 1 μL of template plasmid and 0.5 L of each of forward and reverse primers in a total volume of 30 μL. PCR amplification conditions were as follows: 95° C. for 5 minutes, followed by 30 cycles (90° C. for 30 seconds, 55° C.-65° C. for 30 seconds, and 72° C. for 5 minutes), and 72° C. for 10 minutes.

At the end of PCR, gel electrophoresis analysis, template digestion and transformation were performed with reference to Example 2. Then, cells were harvested with reference to Example 1. Finally, positive clones were screened with reference to Example 3. A mutant at the position 239 (specifically L239G) was obtained.

### Example 6: Comparison of Enzyme Activity of DAAO Mutant II

Combined re-screening was performed on the positive clones screened in Example 5 as described in Example 4, and catalytic efficiency between mutants was compared, as shown in Table 6:

TABLE 6

| Enzyme | | Product | Conversion |
|---|---|---|---|
| No. | Mutant | PPO/mM | Rate (%) |
| 9 | I (M213S/F58H/N54V) | 8.39 ± 0.04 | 33.4 |
| 12 | II (M213S/F58H/ | 12.56 ± 0.04 | 50.24 |
| | N54V/L239G) | | |

As shown in the above table, the activity of the mutant II (M213S/F58H/N54V/L239G) was improved significantly, and subsequently, the modification of thermal stability would be performed through mutation on the basis of the mutant II.

### Example 7: Construction of DAAO Mutant III and Comparison of Enzyme Activity and Thermal Stability

Mutations at positions 73, 77, 79, 147 and 185 were found on the basis of a sequence of the mutant II in Example 6, resulting in a mutant III (M213S/F58H/N54V/L239G/A73L/Q77W/V79M/Q147M/S185M) specifically. Primer sequences for mutant PCR were designed targeting the sites of the mutant III, as shown in Table 7:

TABLE 7

| Serial | Primer | |
|---|---|---|
| Number | Name | Primer Sequence |
| 1 | A73L F | TGGGAGACCTTAACCTTTAACCAGTGGGTTG |
| 2 | A73L R | GGTTAAAGGTTAAGGTCTCCCATTTCGCCTG |
| 3 | 77W/79M F | GACCTTTAACTGGTGGATGGATCTGGTGCCGCAAG |
| 4 | 77W/79M R | GCACCAGATCCATCCACCAGTTAAAGGTCGCGGTC |
| | | TC |
| 5 | Q147M F | GTTCTGCCAGTATCTGATGCGTGAAGCGCAG |
| 6 | Q147M R | GCGCTTCACGATGCAGATACTGGCAGAACT |
| 7 | S185M F | GGGTGCGAAGATGATTGCGGGTGTTGAAGA |
| 8 | S185M R | ACACCCGCAATCATCTTCGCACCCAGACCGGT |

A PCR amplification system and PCR amplification conditions were consistent with those as described in Example 2, and cells were obtained as described in Example 1. The catalytic efficiency of the mutant was detected as described in Example 4. The thermal stability of the mutant was detected by determining the remaining enzyme activity after maintaining a specific temperature for certain time. Specifically, after maintaining at 45° C. for 30 minutes, the remaining enzyme activity was detected by reacting at 30° C. for 1 hour. The catalytic efficiency and the thermal stability effect between single-point and combined mutants were shown in Table 8:

TABLE 8

| | | | | Remaining |
|---|---|---|---|---|
| Enzyme | | Product | Conversion | Enzyme |
| No. | Mutant | PPO/mM | Rate (%) | Activity (%) |
| 12 | II (M213S/F58H/ | 12.58 ± 0.04 | 50.24 | 47.22 |
| | N54V/L239G) | | | |
| 13 | II/A73F | 12.21 ± 0.04 | 48.84 | 70.18 |
| 14 | II/77W/79M | 13.48 ± 0.04 | 53.92 | 83.23 |
| 15 | II/Q147M | 12.53 ± 0.04 | 50.12 | 79.26 |
| 16 | II/S185M | 13.95 ± 0.04 | 55.8 | 92.53 |
| 17 | III (II/A73F/77W/ | 13.64 ± 0.04 | 54.56 | 95.46 |
| | 79M/Q147M/S185M) | | | |

### Example 8: Construction of DAAO Mutant IV and Comparison of Enzyme Activity and Thermal Stability

Mutations at positions 43, 45, 112, 206, 207 and 215 were found on the basis of the sequence of the mutant II in Example 6, resulting in a mutant IV (M213S/F58H/N54V/L239G/A43S/T45M/S206A/D207P/S215F) specifically. Primer sequences designed for mutant PCR targeting the sites of the mutant IV were shown in Table 9:

TABLE 9

| Serial | Primer | |
|---|---|---|
| Number | Name | Primer Sequence |
| 1 | 43/45 F | GATACCGTTTCTCAAATGTTTGCGAGCCCGTGG |
| 2 | 43/45 R | GCTCGCAAACATTTGAGAAACGGTATCTTCCGGC |
| 3 | 206/207 F | TCTGGTGAAGGCTCCTTGCAAACGTTGCACCTCT |
| 4 | 206/207 R | ACGTTTGCAAGGAGCCTTCACCAGAACGGTTTG |
| 5 | 215 F | CACCTCTGACTTTAGCGATCCGAACAGCC |
| 6 | 215 R | CGGATCGCTAAAGTCAGAGGTGCAACGT |

A PCR amplification system and PCR amplification conditions were consistent with those as described in Example 2, and cells were obtained as described in Example 1. The catalytic efficiency of the mutant was detected as described in Example 4. The thermal stability of the mutant was detected by determining the remaining enzyme activity after maintaining a specific temperature for certain time. Specifically, after maintaining at 45° C. for 30 minutes, the remaining enzyme activity was detected by reacting at 30° C. for 1 hour. The catalytic efficiency and the thermal stability effect between single-point and combined mutants were shown in Table 10:

TABLE 10

| | | | | Remaining |
|---|---|---|---|---|
| Enzyme | | Product | Conversion | Enzyme |
| No. | Mutant | PPO/mM | Rate (%) | Activity (%) |
| 12 | II (M213S/F58H/ | 12.58 ± 0.04 | 50.24 | 46.23 |
| | N54V/L239G) | | | |
| 18 | II/A43S/T45M | 13.44 ± 0.03 | 53.76 | 88.84 |
| 19 | II/S206A/D207P | 14.28 ± 0.02 | 57.12 | 92.94 |
| 20 | II/S215F | 13.69 ± 0.03 | 54.76 | 76.96 |
| 21 | IV (II/A43S/T45M/ | 13.78 ± 0.05 | 55.12 | 93.8 |
| | S206A/D207P/S215F) | | | |

### Example 9: Construction of DAAO Mutant V and Comparison of Enzyme Activity and Thermal Stability

Mutations at positions 122, 132, 195 and 234 were found on the basis of the sequence of the mutant II in Example 6, resulting in a mutant IV (M213S/F58H/1N54V/L239G/E122P/Y132F/E195Y/C234L) specifically. Primer sequences designed for mutant PCR targeting the sites of the mutant IV were shown in Table 11:

TABLE 11

| Serial | Primer | |
|---|---|---|
| Number | Name | Primer Sequence |
| 1 | 122 F | GAGAGCAGCCCTTGCCCGCCGGGTGCGATT |
| 2 | 122 R | CCGGCGGGCAAGGGCTGCTCTCCAGTTTAC |
| 3 | 132 F | GGTGTTACCTTTGATACCCTGAGCGTGAAC |
| 4 | 132 R | CAGGGTATCAAAGGTAACACCAATCGCAC |
| 5 | 195 F | CAGGAAGTGTATCCGATTCGTGGCCAAAC |
| 6 | 195 R | CACGAATCGGATACACTTCCTGGTCTTCAAC |
| 7 | 234 F | CGAGGTGATCTTAGGTGGCACCTACCTG |
| 8 | 234 R | GGTGCCACCTAAGATCACCTCGCCACC |

A PCR amplification system and PCR amplification conditions were consistent with those as described in Example 2, and cells were obtained as described in Example 1. The catalytic efficiency of the mutant was detected as described in Example 4. The thermal stability of the mutant was detected by determining the remaining enzyme activity after maintaining a specific temperature for certain time. Specifically, after maintaining at 45° C. for 30 minutes, the remaining enzyme activity was detected by reacting at 30° C. for 1 hour. The catalytic efficiency and the thermal stability effect between single-point and combined mutants were shown in Table 12:

TABLE 12

| | | | | Remaining |
|---|---|---|---|---|
| Enzyme | | Product | Conversion | Enzyme |
| No. | Mutant | PPO/mM | Rate (%) | Activity (%) |
| 12 | II (M213S/F58H/ | 12.58 ± 0.04 | 50.24 | 46.35 |
| | N54V/L239G) | | | |
| 22 | II/E122P | 12.64 ± 0.04 | 50.56 | 89.24 |
| 23 | II/Y132F | 12.53 ± 0.04 | 50.12 | 92.11 |
| 24 | II/E195Y | 13.23 ± 0.04 | 52.92 | 84.46 |
| 25 | II/C234L | 13.28 ± 0.04 | 53.12 | 93.34 |
| 26 | V (II/E122P/Y132F/ | 13.66 ± 0.04 | 54.64 | 94.82 |
| | E195Y/C234L) | | | |

### Example 10: Construction of DAAO Mutant VI and Comparison of Enzyme Activity and Thermal Stability

The above DAAO mutants I-V were subjected to combined mutation to obtain a mutant VI (M213S/F58H/N54V/L239G/A73L/Q77W/V79M/Q147M/S185M/A43S/T45M/S206A/D207P/S215F/E122P/Y132F/E195Y/C234L). A PCR amplification system and PCR amplification conditions were consistent with those as described in Example 2, and cells were obtained as described in Example 1. The catalytic efficiency of the combined mutants II, III, IV, V and VI was detected as described in Example 4. The thermal stability of the mutant was detected by determining the remaining enzyme activity after maintaining a specific temperature for certain time. Specifically, after maintaining at 50° C. for 15 minutes and 55° C. for 15 minutes, respectively, the remaining enzyme activity was detected by reacting at 30° C. for 1 hour. The catalytic efficiency and the thermal stability effect among combined mutants were shown in Table 13:

TABLE 13

| | | Maintain at 50° C. | | Maintain at 55° C. | |
|---|---|---|---|---|---|
| | | for 15 minutes | | for 15 minutes | |
| | | | Remaining | | Remaining |
| | | | Enzyme | | Enzyme |
| Enzyme | | Conversion | Activity | Conversion | Activity |
| No. | Mutant | rate (%) | (%) | rate (%) | (%) |
| 12 | II (M213S/F58H/ | 0 | 0 | 0 | 0 |
| | N54V/L239G) | | | | |
| 17 | III | 72.5 | 82.31 | 64.3 | 46.32 |
| | (II/A73F/Q77W/V79M/ | | | | |
| | Q147M/S185M) | | | | |
| 21 | IV | 79.69 | 74.67 | 67.3 | 50.34 |
| | (II/E122P/T45M/ | | | | |
| | S206A/D207P/S215F) | | | | |
| 26 | V | 69.52 | 81.6 | 59.22 | 53.88 |
| | (II/E122P/Y132F/ | | | | |
| | E195Y/C234L) | | | | |
| 27 | VI | 82.44 | 90.4 | 76.44 | 78.24 |
| | (II/A73L/Q77W/V79M/ | | | | |
| | Q147M/S185M/A43S/T45M/ | | | | |
| | S206A/D207P/S215F/ | | | | |
| | E122P/Y132F/E195Y/C234L) | | | | |

As shown in the above table, the mutant VI exhibited a marked improvement in thermal stability without compromising the enzyme activity. Obviously, the above examples are only used for clearly illustrating examples instead of limiting implementations. Those ordinarily skilled in the art can also make other changes or variations of different forms on the basis of the above illustration. It is unnecessary and impossible to exhaustively list all implementations here. The obvious changes or variations arising therefrom are still within the scope of protection of the present disclosure.

What is claimed is:

1. A D-amino acid oxidase mutant, wherein the D-amino acid oxidase mutant is obtained by carrying out single-point mutation or multi-point combined mutation at positions 54, 58, 213, 239, 73, 77, 79, 147, 185, 43, 45, 206, 207, 215, 122, 132, 195, and 234 of an amino acid sequence as shown in SEQ ID NO. 1.

2. The D-amino acid oxidase mutant according to claim 1, wherein the D-amino acid oxidase mutant comprises one of the following mutations: (1) mutation of an amino acid residue N at the position 54 to V, D or T, mutation of an amino acid residue F at the position 58 to H, R, K or Q, and mutation of an amino acid residue M at the position 213 to S, N or R; (2) mutation of the amino acid residue N at the position 54 to V, D or T, mutation of the amino acid residue F at the position 58 to H, R, K or Q, mutation of the amino acid residue M at the position 213 to S, N or R, and mutation of an amino acid residue L at the position 239 to E, D, G or Q; (3) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at the position 239 to E, D, G or Q, mutation of an amino acid residue A at the position 73 to L, mutation of an amino acid residue Q at the position 77 to W, mutation of an amino acid residue V at the position 79 to M, mutation of an amino acid residue Q at the position 147 to M, and mutation of an amino acid residue S at the position 185 to M; (4) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at the position 239 to E, D, G or Q, mutation of an amino acid residue A at the position 43 to S, mutation of an amino acid residue T at the position 45 to M, mutation of an amino acid residue S at the position 206 to A, mutation of an amino acid residue D at the position 207 to P, and mutation of an amino acid residue S at the position 215 to F; and (5) mutation of the amino acid residue N at the position 54 to V, D or T, mutation of the amino acid residue F at the position 58 to H, R, K or Q, mutation of the amino acid residue M at the position 213 to S, N or R, mutation of the amino acid residue L at position 239 to E, D, G or Q, mutation of an amino acid residue E at the position 122 to P, mutation of an amino acid residue Y at the position 132 to F, mutation of an amino acid residue E at the position 195 to Y, and mutation of an amino acid residue C at the position 234 to L.

3. The D-amino acid oxidase mutant according to claim 2, wherein an amino acid sequence of the mutant is shown in one of SEQ ID NO. 2 to SEQ ID NO. 7.

4. A gene encoding the D-amino acid oxidase mutant according to claim 1.

5. A recombinant vector containing the gene encoding the D-amino acid oxidase mutant according to claim 1.

6. Genetically engineered bacteria containing the gene encoding the D-amino acid oxidase mutant according to claim 1.

7. An application of the D-amino acid oxidase mutant according to claim 1 in preparation of L-phosphinothricin through microbial catalysis.
